(19) United States
(12) Patent Application Publication
(10) Pub. No.: US 2024/0207625 A1
(43) Pub. Date: Jun. 27, 2024

(54) SELF-USE TREATMENT DEVICE

(71) Applicant: Stryker Corporation, Kalamazoo, MI (US)

(72) Inventors: Fred W. Chapman, Newcastle, WA (US); Adam Harvey, Hillsborough (IE)

(73) Assignee: Stryker Corporation, Kalamazoo, MI (US)

(21) Appl. No.: 18/394,633

(22) Filed: Dec. 22, 2023

Related U.S. Application Data
(60) Provisional application No. 63/434,926, filed on Dec. 22, 2022.

(51) Int. Cl. A61N 1/39; A61N 1/04

(52) U.S. Cl. CPC A61N 1/3931; A61N 1/046; A61N 1/3904; A61N 1/3968

(57) ABSTRACT

Patient-operated treatment devices are described herein. An example device detects an electrocardiogram (ECG) of a subject and determines that the subject is operating a medical device. In response to determining that the subject is operating the medical device, the device monitors the subject for consciousness. If the device determines that the subject has become incapacitated while operating the device; it outputs a therapy to the subject.

### CROSS-REFERENCE TO RELATED APPLICATION

This application claims priority to U.S. Provisional App. No. 63/434,926, which was filed on Dec. 22, 2022 and is incorporated by reference herein in its entirety.

### BACKGROUND

An automatic external defibrillator (AED) is an example of a medical device that can be used by an untrained bystander. For example, an AED may instruct a bystander to apply and operate the AED to treat an individual who has collapsed due to ventricular fibrillation (VF). In some examples, the AED automatically assesses the heart rhythm of the individual in order to determine whether to instruct the individual to administer a defibrillating shock to the individual. Due to the unpredictability of VF, and the serious consequences of delaying treatment, many schools, airports, and other places of public accommodation store AEDs in easily accessible locations.

### BRIEF DESCRIPTION OF THE DRAWINGS

FIGS. 1A to 1C illustrate rescue scenes in which an external defibrillator is selectively operated by a patient or a rescuer. FIG. 1A illustrates an environment in which the external defibrillator is operating in a patient-operated mode and the patient is conscious. FIG. 1B illustrates an environment in which the external defibrillator is operating in the patient-operated mode and the patient is unconscious. FIG. 1C illustrates an environment in which the external defibrillator is being operated by the rescuer.

FIG. 2 illustrates an example circuit for detecting whether a subject is conscious.

FIG. 3 illustrates an example electrocardiogram (ECG) that includes a motion artifact.

FIG. 4 illustrates an example of a pulse oximeter configured to confirm whether a subject is conscious.

FIG. 5 illustrates an example of a plethysmograph of a subject that loses a pulse.

FIG. 6 illustrates an example process for automatically administering a treatment based on detected incapacity.

FIG. 7 illustrates an example process for automatically administering a treatment based on the absence of a pulse.

FIG. 8 illustrates an example process for automatically administering a treatment based on the selection of a patient-operated or rescuer-operated mode.

FIG. 9 illustrates an example of an external defibrillator configured to perform various functions described herein.

### DETAILED DESCRIPTION

Various implementations described herein relate to self-operated treatment devices. For instance, an individual may be alone when they experience a serious medical condition, such as ventricular fibrillation (VF). Using devices described herein, the individual may receive a treatment that addresses the serious medical condition without assistance from a bystander. For example, implementations of the present disclosure relate to self-operated defibrillators, such as self-operated AEDs.

In some cases, these devices are configured to operate in different modes. In one mode, the individual experiencing the medical condition operates an example device. For instance, the individual may apply electrodes of a self-use AED to their own chest when they begin to feel symptoms preceding VF (e.g., chest pain, chest pressure, acute shortness of breath, or other heart attack symptoms that can precede VF) or the initial signs of experiencing VF. The self-use AED, for instance, emits an electrical shock to the electrodes in response to detecting the VF, thereby treating the individual. In another mode, another person, such as a bystander or rescuer, operates the device. For instance, if a bystander reaches the individual after they have lost consciousness, the bystander can take over operation of the self-use AED by changing the operating mode of the device.

Implementations of the present disclosure address specific problems in the technical field of medical treatments. Previous treatment devices, such as existing AEDs, require user input to administer treatments (e.g., defibrillating shocks). Thus, a separate user would operate a treatment device in order to treat a patient. However, patients in need of these treatments may not be in the presence of a bystander or rescuer or may otherwise be able to act more quickly to begin a self-treatment than a bystander or rescuer. Accordingly, these devices are unable to treat patients who experience medical emergencies while alone and initiate treatments less quickly than those that can be self-initiated.

While implantable and wearable devices can also administer treatments without separate user intervention, these devices have significant drawbacks. For example, implantable devices are highly invasive and can result in substantial immune responses that can harm patients after placement. Wearable devices can be cumbersome and may be inconvenient for patients. In addition, each implantable or wearable device is associated with a single patient. Without histories of medical problems, it may be inefficient and unnecessarily expensive to designate an implantable or wearable device to an individual patient. However, patients without histories of medical problems can also be in need of medical treatments. For example, individuals may experience VF without significant known existing health problems.

Various implementations of the present disclosure address these and other problems. In various cases, a medical device operates in multiple modes (e.g., at least two modes). When the medical device is operating in a patient-operated mode, it is configured to automatically treat a patient when the patient becomes incapacitated. Unlike existing implantable or wearable devices, the medical device can be stored in a public space and operated by any individual that is in need of treatment. Furthermore, the medical device, in some cases, also operates in a rescuer-operated mode, in which a bystander can control the medical device to administer the treatment to the individual. Thus, various devices described herein can be utilized in the place of conventional AEDs but are optionally operated by the individual being treated.

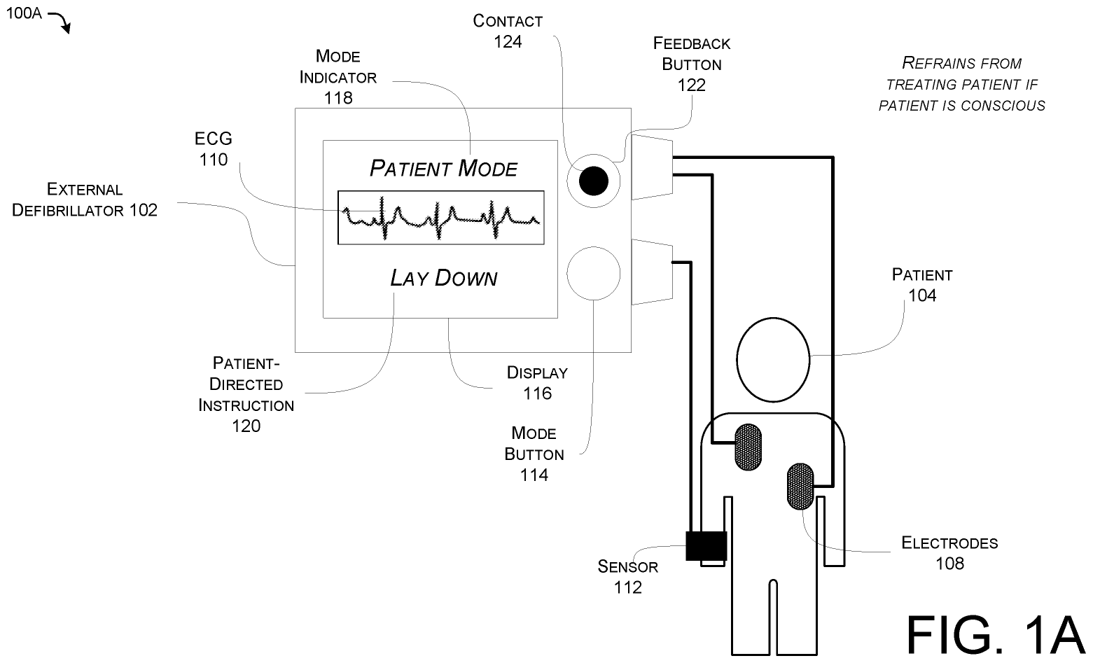

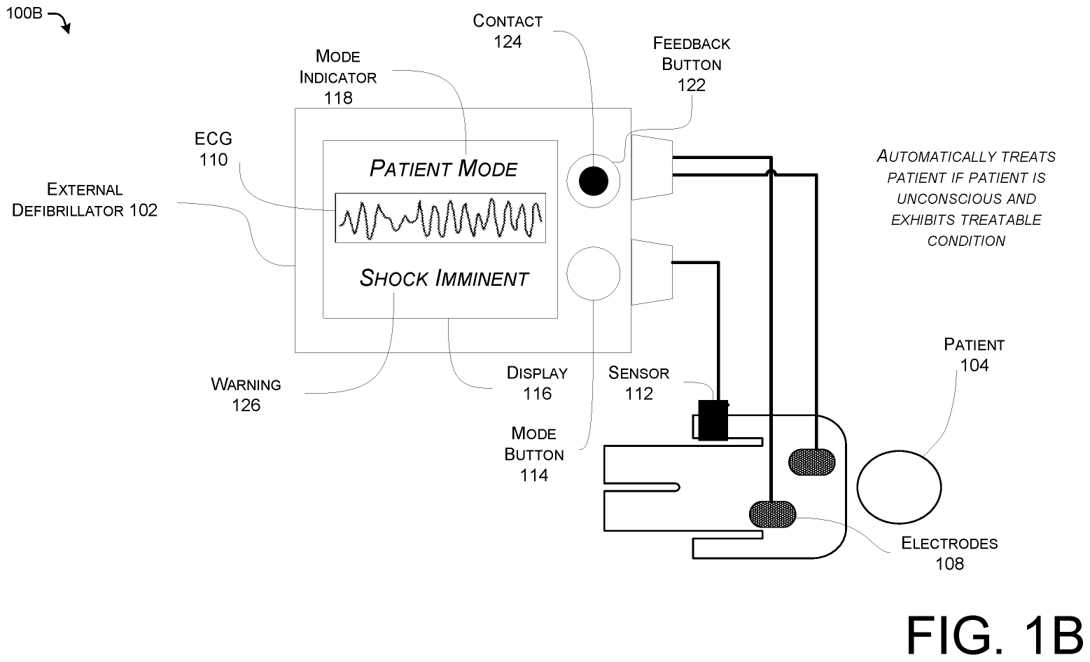

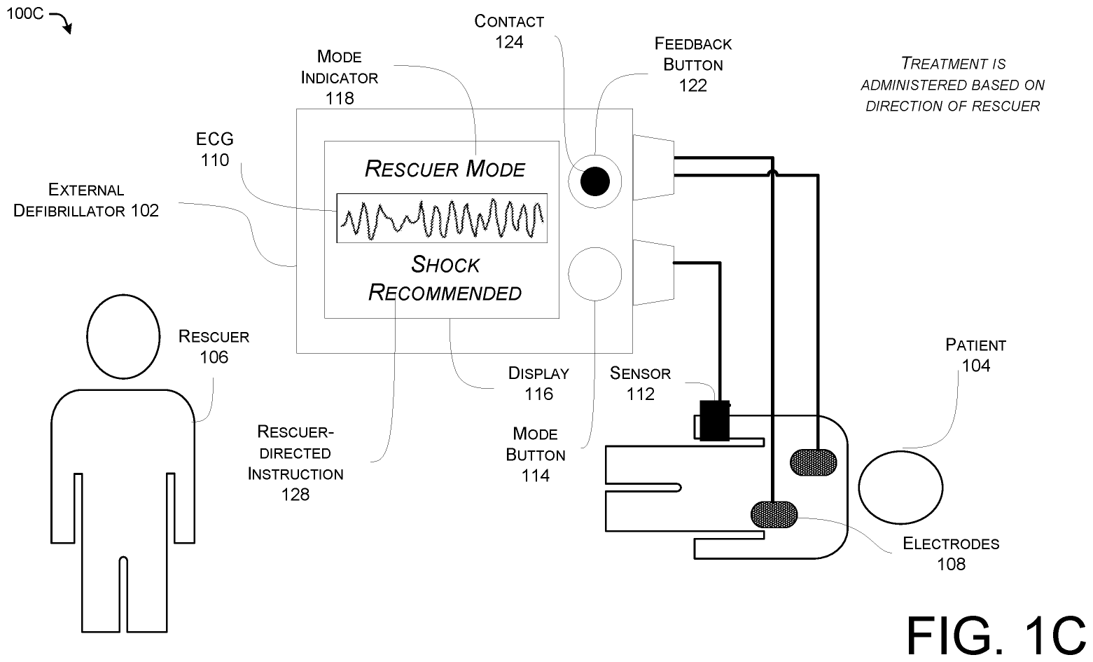

FIGS. 1A to 1C illustrate rescue scenes in which an external defibrillator 102 is selectively operated by a patient 104 or a rescuer 106. In various implementations, the external defibrillator 102 is a dual-mode external defibrillator that operates in at least two modes, such as a patient-operated mode and a rescuer-operated mode. FIG. 1A illustrates an environment 100A in which the external defibrillator 102 is operating in a patient-operated mode and the patient 104 is conscious. In various implementations, the patient 104 may begin operating the external defibrillator 102 in response to self-identifying a potential heart condition. For example, the external defibrillator 102 can be operated by the patient 104 while the patient is conscious. In various implementations, the external defibrillator 102 is configured to treat a variety of individuals, including the patient 104. For instance, unlike many wearable defibrillators, the external defibrillator 102 is not optimized for treating the specific patient 104 described herein. The external defibrillator 102 is not wearable or implantable. In various cases, the external defibrillator 102 is stored in a container that is accessible by the patient 104. For instance, upon feeling light-headed or otherwise unwell, the patient 104 may remove the external defibrillator 102 from the container.

In various cases, the patient 104 self-applies electrodes 108 to the chest of the patient 104. In some examples, the electrodes 108 are adhered to the skin of the patient 104. For example, the electrodes 108 are adhered to the skin of the patient 104 by a biocompatible adhesive. In various cases, the electrodes 108 include a substrate (e.g., a flexible substrate) that is adhered to the skin of the patient 104 by an adhesive. For example, the patient 104 may peel a plastic cover off of the adhesive of the electrodes 108 before self-applying the electrodes 108. The electrodes 108, in various implementations, include an electrically conductive material that is in contact with the skin of the patient 104 when the electrodes 108 are adhered to the chest of the patient 104. Although FIG. 1A depicts two electrodes 108, implementations are not so limited. The electrodes 108, for example, include two electrodes, three electrodes, ten electrodes, or thirteen electrodes. In some examples, the external defibrillator 102 outputs instructions to the patient 104 for applying the electrodes 108. For example, the external defibrillator 102 may communicate the appropriate placement of the electrodes 108 on the chest of the patient 104.

The electrodes 108 are connected to additional circuitry in the external defibrillator 102 by wired connections, wireless connections, or a combination thereof. In various examples, the external defibrillator 102 includes a detection circuit configured to detect an ECG 110 of the patient 104 based on the relative voltages between the electrodes 108. In some cases, the detection circuit includes at least one analog to digital converter (ADC) that converts the relative voltages (representing the ECG 110 in an analog format) into digital data (representing the ECG 110 in a digital format). In some cases, the ECG 110 is defined by multiple channels representing respective relative voltages by different pairs of the electrodes 108. These relative voltages are referred to as “leads.” For instance, the ECG 110 includes a single lead, three leads, twelve leads, or fifteen leads.

In addition, the patient 104 self-applies an additional sensor 112 in various implementations. The sensor 112, for instance, is fastened to the patient 104 and/or held by the patient 104. For example, the sensor 112 is configured to be clamped to an extremity of the patient 104, strapped to an extremity of the patient, wrapped around an extremity of the patient, or fastened to the skin of the patient via an adhesive material. According to some examples, the sensor 112 is configured to be held in a hand of the patient 104, between the teeth of the patient 104, or between other body parts of the patient 104 (e.g., between the head and shoulder, between the ankles, or in an armpit of the patient 104).

The sensor 112 is configured to detect a signal that is analyzed by at least one processor in the external defibrillator 102. In some implementations, the sensor 112 is configured to detect a physiological parameter of the patient 104. For example, the sensor 112 is configured to detect at least one of a blood pressure of the patient 104, an oxygenation (e.g., a peripheral capillary oxygen saturation (SpO₂), regional oxygen saturation (rsO₂), or plethysmograph) of the patient 104, a capnograph of the patient 104, an end-tidal parameter (e.g., an end tidal CO₂) of the patient 104, a mechanical pulse (e.g., on a wrist) of the patient 104, a temperature of the patient 104, a respiration rate of the patient 104, an electroencephalogram (EEG) of the patient 104, blood flow in an artery (or blood vessel) of the patient, or any combination thereof. In some instances, the sensor 112 is configured to detect another type of signal. For instance, the sensor 112 detects a pressure, an acceleration, moisture, sound (such as sound within the patient's body) or an electrical signal (e.g., a voltage or current). Examples of the sensor 112 include a blood pressure cuff, an ultrasound-based blood pressure sensor, an ultrasound-based blood flow velocity sensor, a pulse oximeter, a regional oxygen saturation sensor, a capnography sensor (e.g., a nondispersive infrared sensor (NDIR) CO₂ sensor), a flow sensor, an oxygen sensor, an accelerometer, a thermometer, a pressure sensor, a moisture sensor, a voltmeter, a current sensor, or any combination thereof.

Although FIG. 1A illustrates that the sensor 112 is connected to the external defibrillator 102 via a wire, implementations are not so limited. In some examples, the sensor 112 is a wireless accessory device configured to transmit an electromagnetic signal to the external defibrillator 102 that is indicative of the detected signal. For instance, the sensor 112 communicates with the external defibrillator 102 via a BLUETOOTH™ protocol, a WI-FI™ protocol, a Near-Field Communication (NFC) protocol, or a 3rd Generation Partnership Project (3GPP)-standardized protocol (e.g., Long Term Evolution (LTE), New Radio (NR), etc.).

In various cases, the patient 104 selects the patient-operated mode using an input device, such as a mode button 114. For example, the external defibrillator 102 activates the patient-operated mode in response to detecting that the patient 104 has pressed the mode button 114. In some cases, the patient 104 selects the patient-operated mode using another type of input device. For instance, the external defibrillator 102 activates the patient-operated mode in response to detecting a touch signal on a touch screen, to detecting an audible command spoken by the patient 104, to detecting a position of a physical switch, or to detecting a position of a dial.

While in the patient-operated mode, the external defibrillator 102 outputs information to the patient 104. For instance, the external defibrillator 102 includes a display 116 that visually outputs information to the patient 104. According to various cases, the display 116 includes a liquid crystal display (LCD), a light-emitting diode (LED) display, an organic light-emitting diode (OLED) display, or a quantum dot display. In some implementations, the display 116 is a touchscreen that includes one or more touch sensors integrated with the rest of the display 116. For instance, the external defibrillator 102 is configured to receive an input signal by detecting a touch on the display 116.

In various cases, the display 116 visually outputs a mode indicator 118. For instance, the mode indicator 118 is a visual signal that indicates that the external defibrillator 102 is in the patient-operated mode. In some cases, the mode indicator 118 includes words, colors, shapes, or a combination thereof that convey that the external defibrillator 102 is in the patient-operated mode.

The display 116, for example, visually outputs the ECG 110 detected by the external defibrillator 102. In various implementations, the ECG 110 is displayed in real-time or near real-time as the external defibrillator 102 detects the electrical potential between the electrodes 108. For example, the display 116 outputs the ECG 110 such that voltage is defined on a vertical axis and time is defined on a horizontal axis. The ECG 110 output by the display 116 is updated as time progresses, for instance.

In some cases, the display 116 visually outputs a patient-directed instruction 120. The patient-directed instruction 120, for instance, makes recommendations to the patient 104 for operating the external defibrillator 102 and/or minimizing danger to the patient 104. In some cases, the patient-directed instruction indicates how the patient 104 should place the electrodes 108, how the patient 104 should apply the sensor 112, or the like. In some implementations, the patient-directed instruction 120 instructs the patient 104 to take actions that maximize the safety of the patient 104, particularly if the patient 104 loses consciousness. For instance, the patient-directed instruction 120 tells the patient 104 to sit or lay down, to take deep breaths, to elevate the legs of the patient 104, to make a telephone call to a friend or to emergency services, or the like. In some instances, the patient-directed instruction 120 includes a message instructing the patient 104 to take a dosage of aspirin, for instance, if the patient 104 is exhibiting heart attack symptoms.

In various implementations, the external defibrillator 102 is configured to output one or more readiness indicators. For instance, the external defibrillator 102 may be associated with a recommended maintenance frequency. The external defibrillator 102, upon determining that a time period since a last maintenance event exceeds a threshold time period, may output a recommendation to engage in maintenance services before use. In some examples, the external defibrillator outputs the recommendation on the display 116 and/or transmits an indication of the recommendation to an external device.

In various cases, the external defibrillator 102 is configured to determine whether the patient 104 has a treatable condition by analyzing the ECG 110 and/or the signal detected by the sensor 112. The external defibrillator 102 may be configured to resolve the treatable condition by administering a treatment to the patient 104. Examples of treatable conditions include heart arrhythmias (e.g., bradycardia, tachycardia, ventricular fibrillation (VF), pulseless ventricular tachycardia (VT), or asystole), lack of a pulse (i.e., lack of pulsatile blood flow through at least one blood vessel of the patient 104), lack of breathing (also referred to as “apnea”), or seizure. Further, the external defibrillator 102 is configured to administer a treatment to the patient 104 in response to detecting the treatable condition. Examples of treatments include administering pace pulses via the electrodes 108, administering an electrical shock via the electrodes 108 (e.g., to defibrillate the patient 104), administering chest compressions to the patient 104 (e.g., by controlling a mechanical chest compression device, such as LUCAS™ from Stryker Corporation, of Kalamazoo, MI), administering assisted ventilation to the patient 104 (e.g., by controlling a ventilation device), or administering a medication to the patient 104 (e.g., by controlling a device connected intravenously to the patient 104).

In some implementations, the external defibrillator 102 is configured to output pace pulses to the electrodes 108 in response to determining that the patient 104 has less than a threshold heart rate and/or pulse rate. That is, the external defibrillator 102 may determine that the patient 104 has bradycardia. For example, the external defibrillator 102 determines the heart rate of the patient 104 based on the ECG and/or determines the pulse rate of the patient 104 based on the plethysmograph of the patient 104. The pace pulses, in some implementations, cause the heart rate and/or pulse rate of the patient 104 to increase above the threshold. In some cases, the external defibrillator 102 is configured to output pace pulses to the electrodes 108 only if the patient 104 is unresponsive, unconscious, or obtunded.

According to various implementations, the external defibrillator 102 is configured to output an electrical shock to the electrodes 108 in response to determining that the patient 104 is experiencing a shockable heart arrhythmia. As used herein, the terms “shockable arrhythmia,” “shockable heart arrhythmia,” “shockable rhythm,” and their equivalents, may refer to a heart rhythm that can be resolved by the application of an electrical shock to the heart, such as VF or pulseless VT. The process of applying the electrical shock to resolve the shockable arrhythmia is referred to as “defibrillation.”

When the heart of the patient 104 is exhibiting a shockable arrhythmia, the heart may be unable to mechanically pump blood effectively through the body of the patient 104. The insufficient transport of blood through the body of the patient 104 can prevent the brain and other vital organs from receiving sufficient oxygenation, which can cause the patient 104 to become obtunded and/or lose consciousness. Thus, the external defibrillator 102 may be configured to administer the electrical shock to the patient 104, particularly when the patient 104 is unconscious. As used herein, the term “obtunded,” and its equivalents, may refer to a state of lethargy or reduced consciousness. As used herein, the term “unconscious,” and its equivalents, may refer to a state of not being awake. As used herein, the term “incapacitated,” and its equivalents, may refer to a state of being unable to operate a device, such as the external defibrillator 102. For example, if the patient 104 is unconscious, they are incapacitated. However, the patient 104 may be incapacitated but may remain conscious.

The external defibrillator 102 may automatically administer the electrical shock to the patient 104, without necessarily receiving an input signal indicating that the patient 104 or some other operator has selected the treatment. In some implementations, the external defibrillator 102 outputs a warning signal, such as an audible alarm, before administering the electrical shock. This audible alarm may warn anyone who enters the vicinity of the external defibrillator 102 to avoid contact with the patient 104, in order to avoid receiving the electrical shock.

In some examples, the patient 104 retains consciousness for some time after initially presenting the treatable condition (e.g., the shockable arrhythmia). For example, the patient 104 may be conscious for seconds to minutes after the shockable arrhythmia begins. If, however, the external defibrillator 102 outputs the electrical shock to the electrodes 108 before the patient 104 loses consciousness, the electrical shock can be very painful to the patient 104. Furthermore, if the external defibrillator 102 administers the electrical shock in response to mistakenly identifying the shockable arrhythmia in the ECG 110 and/or the shockable arrhythmia naturally resolves without the electrical shock, the external defibrillator 102 can cause significant harm to the patient 104 by administering the electrical shock.

In various implementations, even if the external defibrillator 102 determines that the patient 104 exhibits the treatable condition, the external defibrillator 102 refrains from administering the treatment to the patient 104 if the external defibrillator 102 detects at least one condition indicating that the patient 104 is conscious. For example, the external defibrillator 102 is prevented from administering an electrical shock if the patient 104 is conscious.

According to some examples, the external defibrillator 102 detects that the patient 104 is conscious by analyzing the ECG 110. In some cases, movement of the patient 104 generates an artifact in the ECG 110. For example, the movement indicates that the patient 104 is conscious. Thus, the external defibrillator 102 may detect that the patient 104 is conscious by detecting a movement artifact in the ECG 110. In some cases, in response to detecting the movement artifact, the external defibrillator 102 may also output an instruction to the patient 104 to be still for a time period (e.g., a 30-second time period) and/or may otherwise output an audible instruction at lower than a threshold volume to induce the patient 104 to be still (e.g., in order to hear the instruction). In some cases, the external defibrillator 102 may perform one or more analyses on a segment of the ECG 110 obtained during this time period. The segment may have minimal motion artifact, such that the signal quality of the segment may be greater than in a segment of the ECG 110 that includes a motion artifact. In some cases, the external defibrillator 102 determines whether the shockable arrhythmia is present in the segment of the ECG 110 detected during the time period.

Not all movement artifacts in the ECG 110 are necessarily indicative that the patient 104 is conscious. For example, chest compressions administered by a bystander or a mechanical chest compression device would produce a movement artifact in the ECG 110, even if the patient 104 is unconscious. In various implementations, the external defibrillator 102 confirms that the patient 104 is conscious by determining that the movement artifact in the ECG 110 is not caused by chest compressions administered to the patient 104. For example, the external defibrillator 102 determines that the movement artifact is non-periodic.

In some instances, the external defibrillator 102 determines that the patient is conscious by determining that the movement artifact in the ECG 110 corresponds to another type of input signal detected by the external defibrillator 102 or the sensor 112. For instance, the external defibrillator 102 determines that the movement artifact occurs simultaneously with the external defibrillator 102 detecting that the mode button 114 has been pushed, that the feedback button 122 has been pushed, or that the display 116 has detected a touch signal.

In some cases, the external defibrillator 102 detects that the patient 104 is conscious by analyzing a signal detected by the sensor 112. In some implementations, an acceleration detected by the sensor 112 indicates that the patient 104 is conscious. For instance, the external defibrillator 102 determines that a motion artifact in the ECG 110 occurs simultaneously with an acceleration of a foot or a hand of the patient 104, as detected by the sensor 112.

In various instances, the external defibrillator 102 detects that the patient 104 is conscious based on an input signal detected by an input device, such as a feedback button 122. For example, if the patient 104 is pressing the feedback button 122, the external defibrillator 102 infers that the patient 104 is conscious. In some implementations, the patient 104 may be instructed (e.g., via the patient-directed instruction 120) to continuously press the feedback button 122 while the patient 104 is conscious.

In some cases, the external defibrillator 102 detects that the patient 104 is conscious based on an electrical signal detected by a contact 124. In various implementations, the external defibrillator 102 is configured to detect an electrical signal at the contact 124. For example, the detection circuit configured to detect the ECG 110 of the patient 104 further detects a relative voltage and/or current between the electrodes 108 and the contact 124. The external defibrillator 102, in some cases, detects that the patient 104 is conscious by determining that the electrical signal is indicative of the patient 104 touching the contact 124 and the electrodes 108 simultaneously. The patient-directed instruction 120, for example, may instruct the patient 104 to touch the contact 124 continuously while the patient 104 is conscious. Although FIG. 1A illustrates the contact 124 as being incorporated into the feedback button 122, implementations are not so limited. For example, the contact 124 may be located on at least one of the electrodes 108 or on the sensor 112.

According to some examples, the external defibrillator 102 infers whether the patient 104 is conscious by monitoring a response of the patient 104 to a test pulse. For example, the external defibrillator 102 may output an electrical signal to the electrodes 108 and monitor a movement of the patient 104 in response to the electrical signal. The movement, for instance, is detected based on a motion artifact in the ECG 110 and/or an acceleration of the patient 104. The presence and/or duration of the movement may be indicative of the consciousness of the patient 104. If the movement is greater than a threshold movement (or if a duration of the movement is less than a threshold duration), the external defibrillator 102 may infer that the patient 104 is conscious. If the movement is less than the threshold movement (or if the duration of the movement is less than the threshold duration), the external defibrillator 102 may infer that the patient 104 is unconscious.

The external defibrillator 102, in various examples, utilizes other techniques for detecting that the patient 104 is conscious. For example, the external defibrillator 102 includes a microphone configured to detect a sound in the environment 100A. The external defibrillator 102 detects that the patient 104 is conscious in response to detecting at least a component of the sound that is consistent with the voice of the patient 104 and/or one or more words indicating that someone within the environment 100A reports that the patient 104 is conscious. In some implementations, the external defibrillator 102 may determine that the patient 104 is unconscious if the microphone detects a sound indicative of agonal respirations. For example, the external defibrillator 102 may determine whether a spectral signature consistent with the sound of agonal respiration is present in a spectra representative of the detected sound. Agonal respirations are an indication of unconsciousness and may be indicative of a good prognosis if the patient 104 is brought back to consciousness relatively quickly.

While in the patient-operated mode, the external defibrillator 102 may perform additional actions to assist the patient 104. In some implementations, the external defibrillator 102 may initiate communications between the patient 104 and emergency services. For instance, the external defibrillator 102 may recommend, or directly initiate, a communication session with an emergency services operator (e.g., using 911). The emergency services provider, for instance, may dispatch an ambulance or emergency medical technician team to assist the patient 104. In some implementations, another type of emergency services device is the recipient of communications with the external defibrillator 102. For example, the emergency services device could be operated by a response office that can be contacted via a subscription plan associated with the external defibrillator 102.

In some implementations, the external defibrillator 102 sends data indicative of the ECG or other physiological parameter(s) to an external device, which may be operated by a care provider in a clinical environment that the patient 104 is later transported (e.g., via the ambulance). In some cases, the external defibrillator 102 recommends, or directly initiates, a communication session with an emergency contact of the patient 104, such as a friend or family member. In various cases, in response to determining that the patient 104 has a treatable condition and/or is unconscious, the external defibrillator 102 may automatically unlock doors to a building where the patient 104 is located (e.g., a home of the patient 104). For example, the locks of the building may be communicatively coupled to a computing device, such as a smart home or Internet of Things (IoT) device. The external defibrillator 102 may cause the computing device to disengage the locks of the building by transmitting an instruction to the computing device. Thus, emergency medical personnel who are dispatched to the building, for instance, may therefore be able to efficiently access the patient 104 for additional support.

In some cases, the external defibrillator 102 communicates directly with an external device (e.g., an emergency services device, a remote server, an external device operated by a clinical provider, or the like). In some examples, the external defibrillator 102 communicates via an intermediary device. For example, the external defibrillator 102 may transmit communication signals directly to an intermediary device, such as a wearable device worn by the patient 104 (e.g., a smart watch worn by the patient 104) or some other device on the patient 104 (e.g., a smart phone in a pocket of the patient 104), such as via a short-range wireless communications interface (e.g., a BLUETOOTH interface). The intermediary device, in various cases, may communicate with the external device that is the recipient of the data in the communication signals. In some examples, the intermediary device communicates over one or more communication interfaces, such as a cellular communication interface.

FIG. 1B illustrates an environment 100B in which the external defibrillator 102 is operating in the patient-operated mode and the patient 104 is unconscious. In various implementations, the external defibrillator 102 administers the treatment (e.g., an electrical shock) to the patient 104 in response to detecting that the patient 104 exhibits the treatable condition and detecting that the patient 104 is unconscious. FIG. 1B illustrates an example of the external defibrillator 102 configured to treat the patient 104 without the assistance of another user operating the external defibrillator 102. In some implementations, the environment 100B occurs at a time after the patient 104 operates the external defibrillator 102 in the environment 100A. As shown in FIG. 1B, the mode indicator 118 remains indicating that the external defibrillator is in the patient-operated mode.

In various implementations, the external defibrillator 102 determines that the patient 104 is exhibiting the treatable condition. For example, the external defibrillator 102 determines that the ECG 110 exhibits a shockable arrhythmia, such as VF or pulseless VT. In some implementations, the external defibrillator 102 determines that the patient 104 is exhibiting the treatable condition by comparing a segment of the ECG 110 and/or the signal detected by the sensor 112 when the patient 104 was conscious to a segment of the ECG 110 and/or the signal when the patient 104 is unconscious. By comparing the applicable signals, the external defibrillator 102 may infer, with greater certainty, that the patient 104 is exhibiting the treatable condition. That is, the consciousness of the patient 104 may be an additional factor for ascertaining whether the patient 104 has the treatable condition. For example, if a first segment of the ECG 110 detected while the patient 104 was conscious omits an artifact that may be a characteristic of VF, and a segment of the ECG 110 detected after the patient 104 is unconscious includes the artifact, then the external defibrillator 102 may be able to infer that the patient 104 has VF with greater certainty than merely analyzing the second segment of the ECG, alone.

Additionally, the external defibrillator 102 determines that the patient 104 is unconscious. For example, the external defibrillator 102 determines that a motion artifact (that is indicative of the patient 104 being conscious) is absent from the ECG 110. In various implementations, the external defibrillator 102 determines that it has not received an input signal from one or more input devices for a threshold period of time. For example, the external defibrillator 102 determines that an input signal has not been detected by the display 116, the mode button 114, the feedback button 122, the contact 124, or any combination thereof.

According to some examples, the external defibrillator 102 infers that the patient 104 is unconscious by determining that the patient 104 does not have a pulse. As used herein, the term “pulse,” and its equivalents, can refer to a periodic flow of blood through at least one blood vessel. In various cases, the sensor 112 includes an oxygenation sensor configured to detect a plethysmograph (or “pleth”) waveform of the patient 104. The external defibrillator 102, in some cases, determines whether the patient 104 has a pulse by analyzing the plethysmograph. In some cases, the external defibrillator 102 determines whether the patient 104 has a pulse based on a blood pressure of the patient 104, which may be detected by the sensor 112. For instance, the external defibrillator 102 determines that a systolic and/or diastolic blood pressure of the patient 104 is above a threshold. If the patient 104 has pulsatile blood flow, it may be unlikely that the patient 104 has lost consciousness due to a shockable arrhythmia, because the pulse may indicate that the brain of the patient 104 is receiving oxygenated blood.

In various cases, the external defibrillator 102 infers that the patient 104 is unconscious by detecting that the patient 104 has not exhibited breathing for greater than a threshold amount of time (e.g., one minute). For example, the sensor 112 detects a respiratory parameter (e.g., a respiration rate, a capnograph, or an EtCO₂) of the patient 104, and the external defibrillator 102 determines the patient 104 is not breathing by comparing the respiratory parameter to a threshold. In some cases, the external defibrillator 102 relies on other factors, in combination with whether the patient 104 is breathing, to assess the consciousness of the patient 104.

In some implementations, the external defibrillator 102 determines that the patient 104 is unconscious by detecting that the patient 104 is exhibiting agonal respirations. For example, the external defibrillator 102 or the sensor 112 includes a microphone configured to detect respiratory sounds from the patient 104. If the external defibrillator 102 determines detected respiratory sounds are indicative of agonal respirations, then the external defibrillator 102 may determine that the patient 104 is unconscious and/or in cardiac arrest.

Once the external defibrillator 102 determines that the patient 104 exhibits the treatable condition and is unconscious, the external defibrillator 102 may begin preparing to administer the treatment. In various implementations, the external defibrillator 102 delays administering the treatment for a pause period after identifying that the patient 104 exhibits the treatable condition and is unconscious. In some cases, the external defibrillator 102 delays the treatment for seconds to minutes (e.g., 2 seconds to 2 minutes). For instance, the external defibrillator 102 charges a capacitor with energy to be output as the electrical shock during the pause period.

In various cases, the display 116 visually outputs a warning 126 indicating that the treatment will be administered to the patient 104. In some cases, the warning 126 further indicates that the treatment will be administered unless the external defibrillator 102 receives an input signal that indicates the patient 104 is conscious, or that otherwise indicates that the treatment should be halted, within the pause period. For example, if the patient 104 regains consciousness and presses the feedback button 122 during the pause period, the external defibrillator 102 may refrain from administering the treatment.

If the pause period expires without the external defibrillator 102 detecting evidence that the patient 104 is conscious, the external defibrillator 102 automatically administers the treatment. For example, the external defibrillator 102 administers a biphasic electrical shock to the heart of the patient 104 via the electrodes 108. Thus, the external defibrillator 102 can treat a shockable arrhythmia of the patient 104 even when the patient 104 is alone and unconscious.

In some implementations, the external defibrillator 102 administers multiple treatments to the patient 104 while the patient is unconscious. For instance, the external defibrillator 102 may administer an electrical shock to the patient 104 via the electrodes. However, if the external defibrillator 102 determines that the patient 104 is still incapacitated (e.g., unconscious) greater than a threshold time period after administering the electrical shock, the external defibrillator 102 may further administer pace pulses to the patient 104 and/or cause administration of chest compressions to the patient 104. The threshold time period, for instance can be between 1 second and 1 minute.

In various cases, the external defibrillator 102 activates the patient-operated mode in response to determining that the electrodes 108 and the sensor 112 are applied to the same person (i.e., the patient 104). This may reduce the risk of improperly treating (or refraining from treating) the patient 104 in response to a signal detected by the sensor 112 from another person, such as a bystander. In various implementations, the external defibrillator 102 determines that the electrodes 108 and the sensor 112 are applied to the same person by determining that the ECG 110 is synchronized with a signal detected by the sensor 112. For instance, the external defibrillator 102 determines that QRS complexes indicated by the ECG 110 are temporally synchronized with a pulse indicated by the signal detected by the sensor 112 (e.g., an spO₂ signal).

FIG. 1C illustrates an environment 1000 in which the external defibrillator 102 is being operated by the rescuer 106. For example, the rescuer 106 may arrive at the environment 1000 when the patient 104 is unconscious or after the patient has begun to operate the external defibrillator 102.

In various implementations, the external defibrillator 102 activates the rescuer-operated mode in response to detecting the rescuer 106. For example, the rescuer 106 selects the rescuer-operated mode by operating the mode button 114. As used herein, the term “rescuer-operated mode,” “bystander-operated mode,” “third-party-operated mode,” and their equivalents, may refer to a state of a treatment device that can be operated by an individual who is not the person being treated. For example, the treatment device can be operated by a rescuer. In some implementations, the external defibrillator 102 initiates the rescuer-operated mode in response to detecting that the rescuer 106 is touching the contact 124. For instance, the detection circuit determines that a different person than the patient 104 is touching the contact 124 by comparing the electrical signal detected by the contact 124 to the ECG 110 detected by the electrodes 108. In some cases, the external defibrillator 102 infers that the rescuer 106 is present in response to detecting that an input signal has been detected at the mode button 114, the display 116, the feedback button 122, or the contact 124 without detecting a motion artifact in the ECG 110 (or an acceleration at the sensor 112). The external defibrillator 102 indicates activation of the rescuer-operated mode via the mode indicator 118.

In some implementations, the external defibrillator 102 detects the rescuer 106 in response to detecting manual chest compressions performed on the patient 104. For example, in some cases, the external defibrillator 102 may detect a chest compression artifact in the ECG 110. The shape of the chest compression artifact, in some cases, is indicative of manual chest compressions (e.g., rather than mechanical chest compressions administered from a mechanical chest compression device). In various cases, the external defibrillator 102 may be communicatively coupled to a mechanical chest compression device and may receive a communication signal from the mechanical chest compression device that indicates that the mechanical chest compression device is not performing the chest compressions indicated by the ECG 110. According to various implementations, the external defibrillator 102 may infer the presence of the rescuer 106 based on a chest compression artifact present in the ECG 110 or some other physiological parameter detected by the external defibrillator 102.

While in the rescuer-operated mode, the external defibrillator 102 outputs a rescuer-directed instruction 128. Unlike the patient-directed instruction 120 or the warning 126, the rescuer-directed instruction 128 is targeted to the rescuer 106, rather than the patient 104. In some examples, the rescuer-directed instruction 128 may instruct the rescuer 106 how to operate the external defibrillator 102. For instance, upon detecting that the patient 104 exhibits the treatable condition and is unconscious, the external defibrillator 102 may instruct the rescuer 106 to administer the treatment to the patient 104. In some cases, the rescuer 106 administers the treatment by operating the external defibrillator 102, such as by pressing a button (e.g., the feedback button 122) of the external defibrillator 102. In some implementations, the external defibrillator 102 outputs other types of instructions, such as instructions to check for a pulse of the patient 104, instructions to administer manual chest compressions to the patient 104, or instructions to administer a medication to the patient 104. Various instructions can be visually output on the display 116 and/or audibly output by a speaker.

In the rescuer-operated mode, the external defibrillator 102 refrains from treating the patient 104 unless the external defibrillator 102 detects that the patient 104 has the treatable condition and that the external defibrillator 102 has detected an input signal from the rescuer 106. Thus, in the rescuer-operated mode, the external defibrillator 102 only treats the patient 104 in response to direction from the rescuer 106.

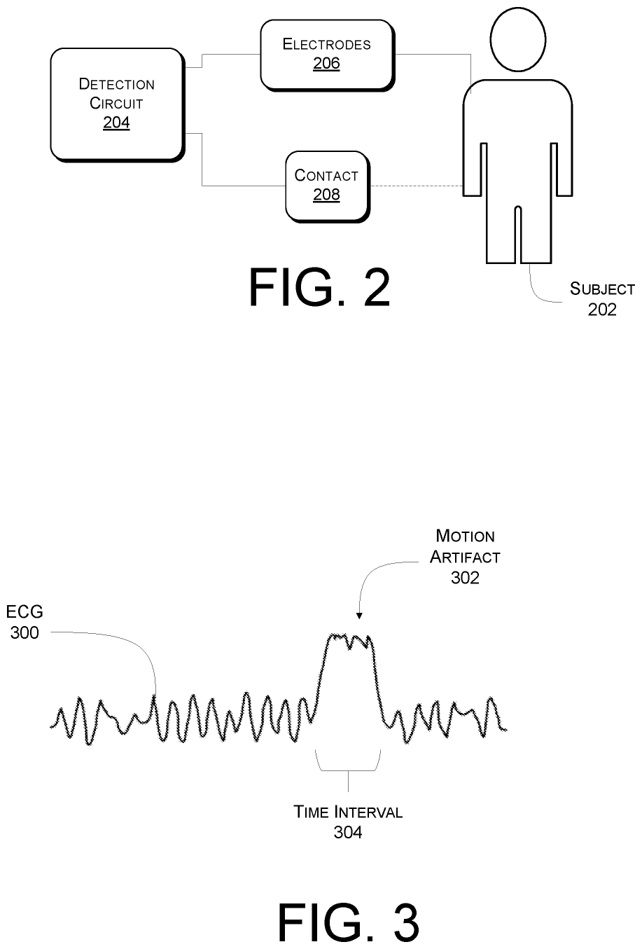

FIG. 2 illustrates an example circuit 200 for detecting whether a subject 202 is conscious. In various implementations, the circuit 200 is at least partially incorporated into a medical device, such as the external defibrillator 102 illustrated in FIGS. 1A-1C, a. In some cases, the subject 202 is the patient 104 described above with reference to FIGS. 1A-1C.

The circuit 200 includes a detection circuit 204. In various implementations, the detection circuit 204 is configured to detect electrical signals. The detection circuit 204 is coupled to electrodes 206 and a contact 208. In some cases, the detection circuit 204 detects an ECG of the subject 202 based on electrical signals received by the electrodes 206. Further, the detection circuit 204 may be configured to detect whether the subject 202 is touching the contact 208 by comparing the electrical signals received by the electrodes 206 and an electrical signal received by the contact 208. For instance, if the subject 202 is touching the electrodes 206 and the contact 208 simultaneously, the relative voltage between the contact 208 and at least one of the electrodes 206 may have a similar periodicity to the ECG of the subject 202, as detected by the electrodes 206.

In various implementations, the medical device refrains from administering a treatment to the subject 202 until the detection circuit 204 detects that the subject 202 is separated from the contact 208. For instance, the medical device may administer an electrical shock to the subject 202 in response to determining that the subject 202 has lost contact with the contact 208.

FIG. 3 illustrates an example ECG 300 that includes a motion artifact 302. In various implementations, the ECG 300 is detected by a medical device, such as the external defibrillator 102 described above with reference to FIGS. 1A-1C. The ECG 300 is detected as a relative voltage over time between electrodes in contact with a subject. For example, the electrodes are adhered to the chest of the subject. Movement of the subject generates the motion artifact 302. In various implementations, the motion artifact 302 is characterized as having a frequency component that is less than a threshold associated with heart rhythms. In some implementations, the external defibrillator detects an electrical impedance of the subject via the electrodes. The external defibrillator, for instance, identifies the motion artifact 302 by determining that the motion artifact 302 is time-synchronized with a corresponding artifact in the impedance.

According to some implementations, the medical device detects that the subject is conscious based on detecting the motion artifact 302. For example, the medical device identifies an input signal (e.g., a touch of a touchscreen, a push of a button, etc.) during a time interval 304 in which the motion artifact 302 occurs. Thus, the medical device infers that the motion artifact 302 corresponds to the motion that enables the subject to provide the input signal, as opposed to another type of motion that the subject may experience while unconscious (e.g., motion due to receiving chest compressions from a rescuer).

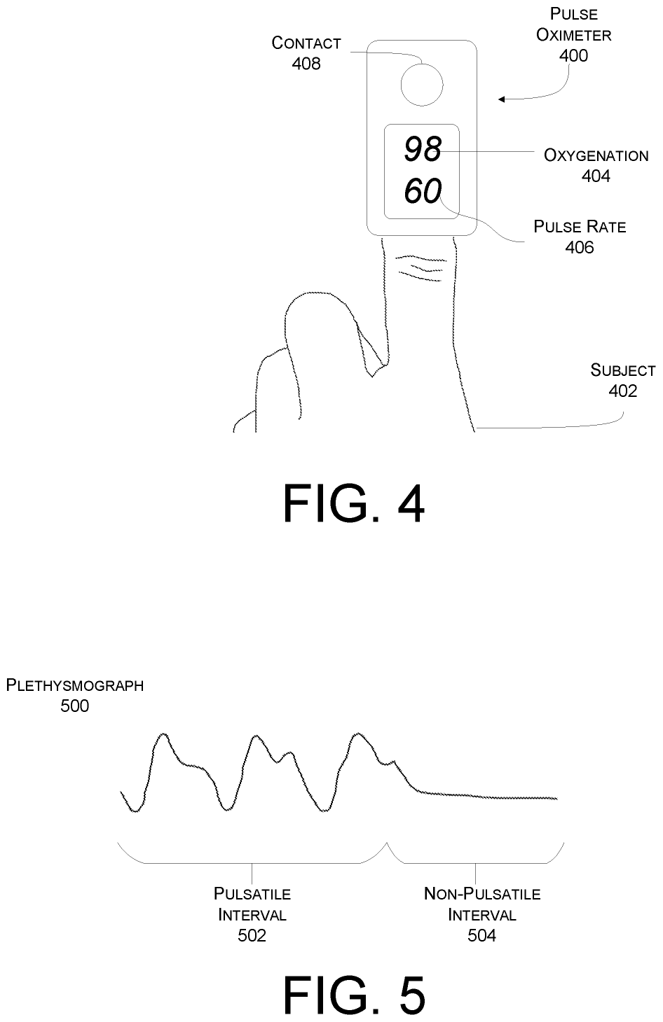

FIG. 4 illustrates an example of a pulse oximeter 400 configured to confirm whether a subject 402 is conscious. In some cases, the pulse oximeter 400 is the sensor 112 described above with reference to FIGS. 1A-1C.

In various implementations, the pulse oximeter 400 includes at least one light source that emits light into an extremity of the subject 402, such as a finger. At least one light sensor detects the light after it is scattered by the extremity. In some cases, the pulse oximeter 400 emits light corresponding to at least two wavelengths, such as wavelengths corresponding to red light and infrared light. The pulse oximeter 400, in some cases, detects the transmission of both wavelengths of light through the extremity of the subject 402. The absorption of the wavelengths of light can be determined based on the transmitted light detected by the pulse oximeter 400.

The pulse oximeter 400 may detect the oxygenation of blood flowing through the extremity of the subject 402 based on the detected light. For example, the pulse oximeter 400 determines the oxygenation based on a ratio of pulse-related oscillations of the transmitted red and infrared light detected by the pulse oximeter 400. In various examples, based on the light detected by the light sensor over time, the pulse oximeter 400 determines the plethysmograph of the subject 402. In some cases, the plethysmograph represents the red or infrared light detected by the pulse oximeter 400 over time. In various implementations, the pulse oximeter 400 determines an oxygenation 404 of the blood of the subject 402 based on the plethysmograph. Further, the pulse oximeter 400 determines a pulse rate 406 of the subject based on the plethysmograph. In various implementations, the pulse oximeter 400 transmits signals indicative of the oxygenation 404 and pulse rate 406 to a medical device.

The pulse oximeter 400 and/or the medical device, in various implementations, determines that the subject 402 has a pulse by analyzing the plethysmograph. For example, the entity determines that the subject 402 has a pulse by determining that the plethysmograph waveform indicates the pulse, such as by determining that the pulse rate 406 is consistent with a pulse. In some cases, the pulse oximeter 400 and/or the medical device determines that the subject 402 has a pulse based on the oxygenation 404 and/or the pulse rate 406. For instance, the pulse oximeter 400 and/or medical device determines that the oxygenation 404 is above a first threshold and/or that the pulse rate 406 is above a second threshold.

The pulse oximeter 400 and/or medical device, in some cases, determines whether the subject 402 is conscious based, at least in part, on determining whether the subject 402 has a pulse. For instance, if the subject 402 does not have a pulse (e.g., for a threshold time period), then the subject 402 is unlikely to be conscious. Notably, the subject 402 may have a pulse and may also be unconscious. Accordingly, in various implementations in which the subject 402 has a pulse, the pulse oximeter 400 and/or the medical device may utilize additional factors to infer whether the subject 402 is unconscious.

In various cases, the pulse oximeter 400 further includes a contact 408. In various implementations, the pulse oximeter 400 detects an electrical signal at the contact 408. The electrical signal is indicative of whether the contact 408 is being touched by the subject 402. The pulse oximeter 400, in some examples, transmits a signal indicative of the detected electrical signal to the medical device. The pulse oximeter 400 and/or the medical device, in some implementations, can detect that the subject 402 is conscious by determining that the subject 402 is touching the contact 408. Further, the pulse oximeter 400 and/or the medical device detect that the subject 402 is unconscious by determining that the subject 402 is separated from the contact 408.

In various implementations, the medical device refrains from administering a treatment to the subject 402 if the subject 402 is determined to have a pulse and/or is conscious. The medical device, in some cases, automatically administers the treatment upon determining that the subject 402 does not have a pulse and/or is unconscious.

It should be noted that in some cases, the medical device administers particular treatments to the subject 402 if the subject 402 is determined to have a pulse. For example, the medical device may administer pace pulses to the subject 402 if the subject 402 is exhibiting bradycardia, which could be indicated and/or otherwise evidenced by a pulse.

FIG. 5 illustrates an example of a plethysmograph 500 of a subject that loses a pulse. For instance, the plethysmograph 500 is detected by the sensor 112 described above with reference to FIGS. 1A-1C. In various implementations, the plethysmograph 500 exhibits a pulse of the subject during a pulsatile interval 502, and exhibits a lack of the pulse during a non-pulsatile interval 504. As shown, the plethysmograph 500 exhibits a periodic waveform with two local maxima, corresponding to the systolic and diastolic phases of blood flowing through the subject. However, when the subject loses the pulse during the non-pulsatile interval 504, the plethysmograph 500 loses the periodicity indicative of pulsatile blood flow. In various implementations, a medical device refrains from administering a treatment during the pulsatile interval 502 and/or administers the treatment to the subject during the non-pulsatile interval 504.

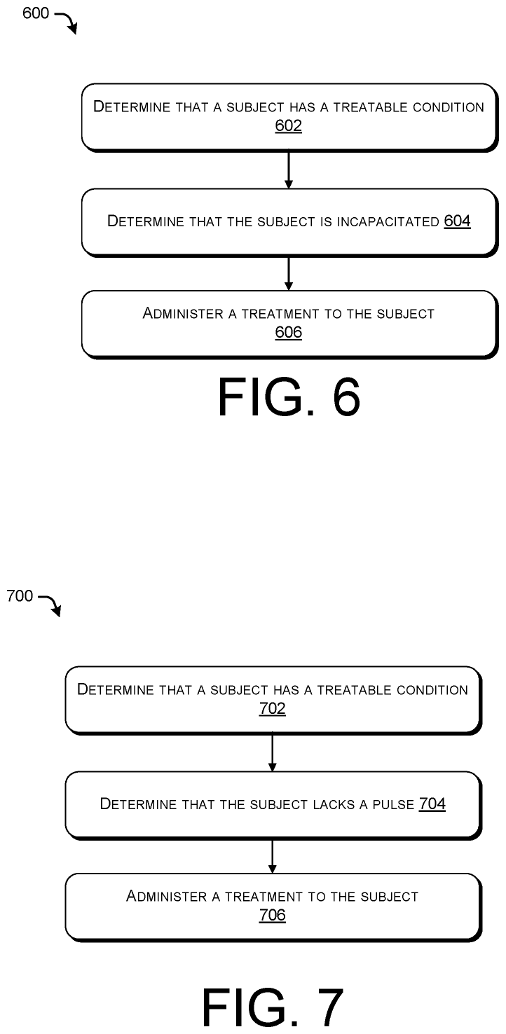

FIG. 6 illustrates an example process 600 for automatically administering a treatment based on detected incapacity. The process 600 is performed by an entity, such as a medical device, a monitor-defibrillator, an intravenous pump, a ventilation device, a mechanical chest compression device, an ultrasound device, or the external defibrillator 102 described above with reference to FIGS. 1A-1C.

At 602, the entity determines that a subject has a treatable condition. In some implementations, the entity determines that the subject is exhibiting at least one of a shockable arrhythmia, bradycardia, a seizure, a lack of breathing, or a lack of pulsatile blood flow. In various implementations, the entity determines that the subject has the treatable condition by analyzing a signal detected from the subject by a sensor. In some cases, the signal is indicative of a physiological parameter. For example, the signal may be indicative of an ECG, a blood pressure, an oxygenation (e.g., SpO₂ or rsO₂), a capnograph, an end-tidal parameter (e.g., an end tidal CO₂), a mechanical pulse, a temperature, a respiration rate, or an EEG of the subject.

Optionally, the entity determines that the subject is operating the entity. For example, the entity detects an input signal indicating that the subject has selected a patient-operated mode of the entity. In some cases, the entity determines that the subject is operating the entity by detecting a motion artifact in the signal detected from the subject by the sensor (e.g., a motion artifact in the ECG). For instance, the entity may determine that the motion artifact is detected simultaneously with an input signal detected by the entity, such as the push of a button or the touch of a touch screen. In some implementations, to enhance certainty of the determination that the subject is operating the entity, the entity may determine that a pattern of motion artifacts is detected simultaneously with a pattern of input signals.

At 604, the entity determines that the subject is incapacitated. In various implementations, the entity determines that the subject is incapacitated in response to determining that an input signal previously detected by the entity has ceased. For example, the entity may determine that the subject is no longer touching an electrical contact by analyzing the ECG of the subject in view of an electrical signal detected by the electrical contact. According to some examples, the entity may determine that a motion artifact (e.g., a non-periodic motion artifact) in the ECG has ceased. In some implementations, the entity may determine that the subject is incapacitated based on a signal detected from the subject by an additional sensor. For example, the entity may determine that an acceleration of a limb of the subject, as detected by an accelerometer, is below a particular threshold. In some cases, the signal is indicative of an additional physiological parameter of the subject. In some cases, the entity may infer that the subject is incapacitated by determining that the subject is not breathing.

At 606, the entity administers a treatment to the subject. In various implementations, the entity administers an electrical shock to electrodes attached to the subject. In some cases, the entity administers pace pulses to the electrodes attached to the subject. Other treatments are also possible. For example, the entity may administer chest compressions to the subject, or may cause a mechanical chest compression device to administer the chest compressions. In some cases, the entity administers assisted ventilation to the subject. According to particular examples, the entity administers a medication to the subject.

FIG. 7 illustrates an example process 700 for automatically administering a treatment based on the absence of a pulse. The process 700 is performed by an entity, such as a medical device, a monitor-defibrillator, an intravenous pump, a ventilation device, an ultrasound device, or the external defibrillator 102 described above with reference to FIGS. 1A-1C.

At 702, the entity determines that a subject has a treatable condition. In some implementations, the entity determines that the subject is exhibiting at least one of a shockable arrhythmia, bradycardia, a seizure, a lack of breathing, or a lack of pulsatile blood flow. In various implementations, the entity determines that the subject has the treatable condition by analyzing a signal detected from the subject by a sensor. In some cases, the signal is indicative of a physiological parameter. For example, the signal may be indicative of an ECG, a blood pressure, an oxygenation (e.g., SpO₂ or rsO₂), a capnograph, an end-tidal parameter (e.g., an end tidal CO₂), a mechanical pulse, a temperature, a respiration rate, or an EEG of the subject.

Optionally, the entity determines that the subject is operating the entity. For example, the entity detects an input signal indicating that the subject has selected a patient-operated mode of the entity. In some cases, the entity determines that the subject is operating the entity by detecting a motion artifact in the signal detected from the subject by the sensor (e.g., a motion artifact in the ECG). For instance, the entity may determine that the motion artifact is detected simultaneously with an input signal detected by the entity, such as the push of a button or the touch of a touch screen. In some implementations, to enhance certainty of the determination that the subject is operating the entity, the entity may determine that a pattern of motion artifacts is detected simultaneously with a pattern of input signals.

At 704, the entity determines that the subject lacks a pulse. For example, an additional sensor detects a signal from the subject and the entity determines that the subject lacks a pulse by analyzing the signal. In some cases, the signal is a plethysmograph waveform detected by a pulse oximeter. In some implementations, the signal is an acceleration detected by an accelerometer. In various cases, the signal is generated by an ultrasound transducer and is indicative of blood flow. For example, the ultrasound transducer may generate the signal by emitting ultrasound toward a blood vessel of the subject, detecting scattered and/or reflected ultrasound from the blood flowing through the blood vessel, and comparing the frequencies of the emitted ultrasound and the detected ultrasound. A difference in the frequencies (also referred to as a “frequency shift”) is indicative of the velocity of the blood. Thus, the entity may determine that the subject has blood flow by performing a Doppler ultrasound technique on the blood vessel of the subject.

At 706, the entity administers a treatment to the subject. In various implementations, the entity administers an electrical shock to electrodes attached to the subject. In some cases, the entity administers pace pulses to the electrodes attached to the subject. Other treatments are also possible. For example, the entity may administer chest compressions to the subject, or may cause a mechanical chest compression device to administer the chest compressions. In some cases, the entity administers assisted ventilation to the subject. According to particular examples, the entity administers a medication to the subject.

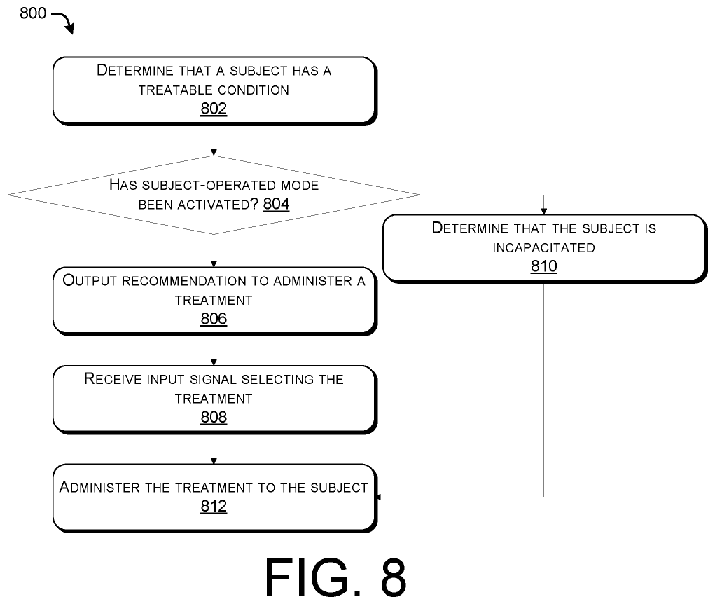

FIG. 8 illustrates an example process 800 for automatically administering a treatment based on the selection of a patient-operated or rescuer-operated mode. The process 800 is performed by an entity, such as a medical device, a monitor-defibrillator, an intravenous pump, a ventilation device, or the external defibrillator 102 described above with reference to FIGS. 1A-1C.

At 802, the entity determines that a subject has a treatable condition. In some implementations, the entity determines that the subject is exhibiting at least one of a shockable arrhythmia, bradycardia, a seizure, a lack of breathing, or a lack of pulsatile blood flow. In various implementations, the entity determines that the subject has the treatable condition by analyzing a signal detected from the subject by a sensor. In some cases, the signal is indicative of a physiological parameter. For example, the signal may be indicative of an ECG, a blood pressure, blood flow (e.g., a blood velocity), an oxygenation (e.g., SpO₂ or rsO₂)), a capnograph, an end-tidal parameter (e.g., an end tidal CO₂), a mechanical pulse, a temperature, a respiration rate, or an EEG of the subject.

At 804, the entity determines whether a subject-operated mode has been activated. For example, the entity detects an input signal indicating that the subject has selected a patient-operated mode of the entity. In some cases, the entity determines that the subject is operating the entity by detecting a motion artifact in the signal detected from the subject by the sensor (e.g., a motion artifact in the ECG). For instance, the entity may determine that the motion artifact is detected simultaneously with an input signal detected by the entity, such as the push of a button or the touch of a touch screen. Alternatively, the entity may determine that the subject-operated mode has not been activated. For instance, the entity detects an input signal indicating that a user (e.g., the subject or a bystander) has selected a bystander-operated mode of the entity. In some implementations, the entity infers that the bystander is operating the entity by observing the absence of a motion artifact in the signal at the time that the entity detects the input signal.

If the entity determines that the subject-operated mode was not activated at 804, then the process 800 proceeds to 806. At 806, the entity outputs a recommendation to administer a treatment. In various implementations, the entity outputs a visual signal indicating the treatment. In some cases, the entity outputs an audible signal indicating the treatment. The entity, for instance, outputs an instruction to administer the treatment to the subject and/or that the subject has the treatable condition.

At 808, the entity receives an input signal selecting the treatment. For example, the entity may detect that a button has been pressed and/or that a touch screen has been touched. The bystander, for instance, provides the input signal to the entity.

If, on the other hand, the entity determines that the subject-operated mode was activated at 804, then the process 800 proceeds to 810. At 810, the entity determines that the subject is incapacitated. In various implementations, the entity determines that the subject is incapacitated in response to determining that an input signal previously detected by the entity has ceased. For example, the entity may determine that the subject is no longer touching an electrical contact by analyzing the ECG of the subject in view of an electrical signal detected by the electrical contact. According to some examples, the entity may determine that a motion artifact (e.g., a non-periodic motion artifact) in the ECG has ceased. In some implementations, the entity determine that the subject is incapacitated based on a signal detected from the subject by an additional sensor. For example, the entity may determine that an acceleration of a limb of the subject, as detected by an accelerometer, is below a particular threshold. In some cases, the signal is indicative of an additional physiological parameter of the subject. In some cases, the entity may infer that the subject is incapacitated by determining that the subject is not breathing. According to some examples, the entity may determine that the subject lacks a pulse. In various implementations, the entity determines that the subject has been incapacitated for greater than a threshold time period, such as 1 second, 2 seconds, 3 seconds, 4 seconds, or 5 seconds.

After performing 808 or 810, the process 800 converges at 812. At 812, the entity administers the treatment to the subject. In various implementations, the entity administers an electrical shock to electrodes attached to the subject. In some cases, the entity administers pace pulses to the electrodes attached to the subject. Other treatments are also possible. For example, the entity may administer chest compressions to the subject, or may cause a mechanical chest compression device to administer the chest compressions. In some cases, the entity administers assisted ventilation to the subject. According to particular examples, the entity administers a medication to the subject.

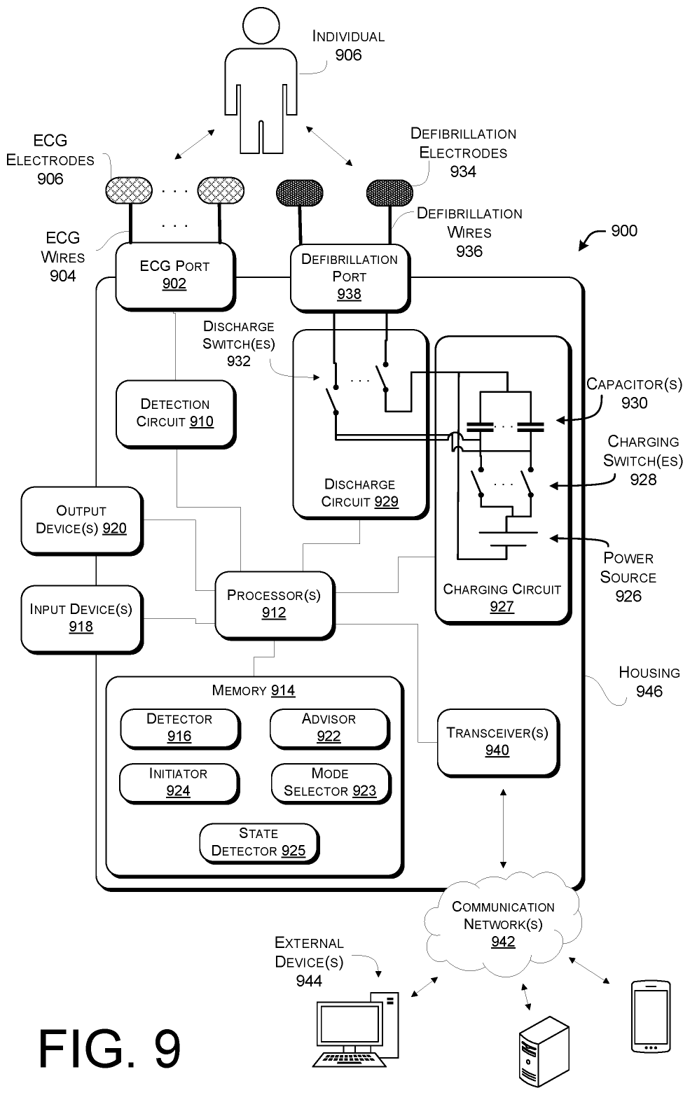

FIG. 9 illustrates an example of an external defibrillator 900 configured to perform various functions described herein. For example, the external defibrillator 900 is the external defibrillator 102 described above with reference to FIGS. 1A-1C.

The external defibrillator 900 includes an electrocardiogram (ECG) port 902 connected to multiple ECG wires 904. In some cases, the ECG wires 904 are removeable from the ECG port 902. For instance, the ECG wires 904 are plugged into the ECG port 902. The ECG wires 904 are connected to ECG electrodes 906, respectively. In various implementations, the ECG electrodes 906 are disposed on different locations on an individual 908. A detection circuit 910 is configured to detect relative voltages between the ECG electrodes 906. These voltages are indicative of the electrical activity of the heart of the individual 908.

In various implementations, the ECG electrodes 906 are in contact with the different locations on the skin of the individual 908. In some examples, a first one of the ECG electrodes 906 is placed on the skin between the heart and right arm of the individual 908, a second one of the ECG electrodes 906 is placed on the skin between the heart and left arm of the individual 908, and a third one of the ECG electrodes 906 is placed on the skin between the heart and a leg (either the left leg or the right leg) of the individual 908. In these examples, the detection circuit 908 is configured to measure the relative voltages between the first, second, and third ECG electrodes 906. Respective pairings of the ECG electrodes 906 are referred to as “leads,” and the voltages between the pairs of ECG electrodes 906 are known as “lead voltages.” In some examples, more than three ECG electrodes 906 are included, such that 5-lead or 12-lead ECG signals are detected by the detection circuit 910.

The detection circuit 910 includes at least one analog circuit, at least one digital circuit, or a combination thereof. The detection circuit 910 receives the analog electrical signals from the ECG electrodes 906, via the ECG port 902 and the ECG wires 904. In some cases, the detection circuit 910 includes one or more analog filters configured to filter noise and/or artifact from the electrical signals. The detection circuit 910 includes an analog-to-digital (ADC) in various examples. The detection circuit 910 generates a digital signal indicative of the analog electrical signals from the ECG electrodes 906. This digital signal can be referred to as an “ECG signal” or an “ECG.”

In some cases, the detection circuit 910 further detects an electrical impedance between at least one pair of the ECG electrodes 906. For example, the detection circuit 910 includes, or otherwise controls, a power source that applies a known voltage (or current) across a pair of the ECG electrodes 906 and detects a resultant current (or voltage) between the pair of the ECG electrodes 906. The impedance is generated based on the applied signal (voltage or current) and the resultant signal (current or voltage). In various cases, the impedance corresponds to respiration of the individual 908, chest compressions performed on the individual 908, and other physiological states of the individual 908. In various examples, the detection circuit 910 includes one or more analog filters configured to filter noise and/or artifact from the resultant signal. The detection circuit 910 generates a digital signal indicative of the impedance using an ADC. This digital signal can be referred to as an “impedance signal” or an “impedance.”

The detection circuit 910 provides the ECG signal and/or the impedance signal one or more processors 912 in the external defibrillator 900. In some implementations, the processor(s) 912 includes a central processing unit (CPU), a graphics processing unit (GPU), both CPU and GPU, or other processing unit or component known in the art.

The processor(s) 912 is operably connected to memory 914. In various implementations, the memory 914 is volatile (such as random access memory (RAM)), non-volatile (such as read only memory (ROM), flash memory, etc.) or some combination of the two. The memory 914 stores instructions that, when executed by the processor(s) 912, causes the processor(s) 912 to perform various operations. In various examples, the memory 914 stores methods, threads, processes, applications, objects, modules, any other sort of executable instruction, or a combination thereof. In some cases, the memory 914 stores files, databases, or a combination thereof. In some examples, the memory 914 includes RAM, ROM, electrically erasable programmable read-only memory (EEPROM), flash memory, or any other memory technology. In some examples, the memory 914 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 912 and/or the external defibrillator 900. In some cases, the memory 914 at least temporarily stores the ECG signal and/or the impedance signal.

In various examples, the memory 914 includes a detector 916, which causes the processor(s) 912 to determine, based on the ECG signal and/or the impedance signal, whether the individual 908 is exhibiting a particular heart rhythm. For instance, the processor(s) 912 determines whether the individual 908 is experiencing a shockable rhythm that is treatable by defibrillation. Examples of shockable rhythms include ventricular fibrillation (VF) and (pulseless) ventricular tachycardia (VT). In some examples, the processor(s) 912 determines whether any of a variety of different rhythms (e.g., asystole, sinus rhythm, atrial fibrillation (AF), etc.) are present in the ECG signal.

The processor(s) 912 is operably connected to one or more input devices 918 and one or more output devices 920. Collectively, the input device(s) 918 and the output device(s) 920 function as an interface between a user and the defibrillator 900. The input device(s) 918 is configured to receive an input from a user and includes at least one of a keypad, a cursor control, a touch-sensitive display, a voice input device (e.g., a speaker), a haptic feedback device, or any combination thereof. The output device(s) 920 includes at least one of a display, a speaker, a haptic output device, a printer, or any combination thereof. In various examples, the processor(s) 912 causes a display among the input device(s) 918 to visually output a waveform of the ECG signal and/or the impedance signal. In some implementations, the input device(s) 918 includes one or more touch sensors, the output device(s) 920 includes a display screen, and the touch sensor(s) are integrated with the display screen. Thus, in some cases, the external defibrillator 900 includes a touchscreen configured to receive user input signal(s) and visually output physiological parameters, such as the ECG signal and/or the impedance signal.

In some examples, the memory 914 includes an advisor 922, which, when executed by the processor(s) 912, causes the processor(s) 912 to generate advice and/or control the output device(s) 920 to output the advice to a user (e.g., a rescuer). In some examples, the processor(s) 912 provides, or causes the output device(s) 920 to provide, an instruction to perform CPR on the individual 908. In some cases, the processor(s) 912 evaluates, based on the ECG signal, the impedance signal, or other physiological parameters, CPR being performed on the individual 908 and causes the output device(s) 920 to provide feedback about the CPR in the instruction. According to some examples, the processor(s) 912, upon identifying that a shockable rhythm is present in the ECG signal, causes the output device(s) 920 to output an instruction and/or recommendation to administer a defibrillation shock to the individual 908.

The memory 914 also includes an initiator 924 which, when executed by the processor(s) 912, causes the processor(s) 912 to control other elements of the external defibrillator 900 in order to administer a defibrillation shock to the individual 908. In some examples, the processor(s) 912 executing the initiator 924 selectively causes the administration of the defibrillation shock based on determining that the individual 908 is exhibiting the shockable rhythm and/or based on an input from a user (received, e.g., by the input device(s) 918. In some cases, the processor(s) 912 causes the defibrillation shock to be output at a particular time, which is determined by the processor(s) 912 based on the ECG signal and/or the impedance signal.

In various implementations, the memory 914 further includes a mode selector 923 and a state detector 925. In various implementations, the mode selector 923, when executed by the processor(s) 912, causes the processor(s) 912 to activate a patient-operated mode or a rescuer-operated mode. Further, the state detector 925, when executed by the processor(s) 912, causes the processor(s) 912 to determine whether the individual 906 is exhibiting a treatable condition (e.g., a shockable arrhythmia), whether the individual 906 is incapacitated, whether the individual 906 is unconscious, whether the individual 906 has a pulse, or the like. In various cases, the processor(s) 912 is configured to cause the defibrillation shock based on the state of the individual 906 identified via the state detector 925.

The processor(s) 912 is operably connected to a charging circuit 927 and a discharge circuit 929. In various implementations, the charging circuit 927 includes a power source 926, one or more charging switches 928, and one or more capacitors 930. The power source 926 includes, for instance, a battery. The processor(s) 912 initiates a defibrillation shock by causing the power source 926 to charge at least one capacitor among the capacitor(s) 930. For example, the processor(s) 912 activates at least one of the charging switch(es) 928 in the charging circuit 927 to complete a first circuit connecting the power source 926 and the capacitor to be charged. Then, the processor(s) 912 causes the discharge circuit 929 to discharge energy stored in the charged capacitor across a pair of defibrillation electrodes 934, which are in contact with the individual 908. For example, the processor(s) 912 deactivates the charging switch(es) 928 completing the first circuit between the capacitor(s) 930 and the power source 926, and activates one or more discharge switches 932 completing a second circuit connecting the charged capacitor 930 and at least a portion of the individual 908 disposed between defibrillation electrodes 934.

The energy is discharged from the defibrillation electrodes 934 in the form of a defibrillation shock. For example, the defibrillation electrodes 934 are connected to the skin of the individual 908 and located at positions on different sides of the heart of the individual 908, such that the defibrillation shock is applied across the heart of the individual 908. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of 0.015 seconds. In some cases, the defibrillation shock has a multiphasic (e.g., biphasic) waveform. The discharge switch(es) 932 are controlled by the processor(s) 912, for example. In various implementations, the defibrillation electrodes 934 are connected to defibrillation wires 936. The defibrillation wires 936 are connected to a defibrillation port 938, in implementations. According to various examples, the defibrillation leads 936 are removable from the defibrillation port 938. For example, the defibrillation wires 936 are plugged into the defibrillation port 938.

In various implementations, the processor(s) 912 is operably connected to one or more transceivers 940 that transmit and/or receive data over one or more communication networks 942. For example, the transceiver(s) 940 includes a network interface card (NIC), a network adapter, a local area network (LAN) adapter, or a physical, virtual, or logical address to connect to the various external devices and/or systems. In various examples, the transceiver(s) 940 includes any sort of wireless transceivers capable of engaging in wireless communication (e.g., radio frequency (RF) communication). For example, the communication network(s) 942 includes one or more wireless networks that include a 3rd Generation Partnership Project (3GPP) network, such as a Long Term Evolution (LTE) radio access network (RAN) (e.g., over one or more LE bands), a New Radio (NR) RAN (e.g., over one or more NR bands), or a combination thereof. In some cases, the transceiver(s) 940 includes other wireless modems, such as a modem for engaging in WI-FI®, WIGIG®, WIMAX®, BLUETOOTH®, or infrared communication over the communication network(s) 942.

The defibrillator 900 is configured to transmit and/or receive data (e.g., ECG data, impedance data, data indicative of one or more detected heart rhythms of the individual 908, data indicative of one or more defibrillation shocks administered to the individual 908, etc.) with one or more external devices 944 via the communication network(s) 942. The external devices 944 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 942. In some examples, the external device(s) 944 is located remotely from the defibrillator 900, such as at a remote clinical environment (e.g., a hospital). According to various implementations, the processor(s) 912 causes the transceiver(s) 940 to transmit data to the external device(s) 944. In some cases, the transceiver(s) 940 receives data from the external device(s) 944 and the transceiver(s) 940 provide the received data to the processor(s) 912 for further analysis.

In various implementations, the external defibrillator 900 also includes a housing 946 that at least partially encloses other elements of the external defibrillator 900. For example, the housing 946 encloses the detection circuit 910, the processor(s) 912, the memory 914, the charging circuit 927, the transceiver(s) 940, or any combination thereof. In some cases, the input device(s) 918 and output device(s) 920 extend from an interior space at least partially surrounded by the housing 946 through a wall of the housing 946. In various examples, the housing 946 acts as a barrier to moisture, electrical interference, and/or dust, thereby protecting various components in the external defibrillator 900 from damage.

In some implementations, the external defibrillator 900 is an automated external defibrillator (AED) operated by an untrained user (e.g., a bystander, layperson, etc.) and can be operated in an automatic mode. In automatic mode, the processor(s) 912 automatically identifies a rhythm in the ECG signal, makes a decision whether to administer a defibrillation shock, charges the capacitor(s) 930, discharges the capacitor(s) 930, or any combination thereof. In some cases, the processor(s) 912 controls the output device(s) 920 to output (e.g., display) a simplified user interface to the untrained user. For example, the processor(s) 912 refrains from causing the output device(s) 920 to display a waveform of the ECG signal and/or the impedance signal to the untrained user, in order to simplify operation of the external defibrillator 900.

In some examples, the external defibrillator 900 is a monitor-defibrillator utilized by a trained user (e.g., a clinician, an emergency responder, etc.) and can be operated in a manual mode or the automatic mode. When the external defibrillator 900 operates in manual mode, the processor(s) 912 cause the output device(s) 920 to display a variety of information that may be relevant to the trained user, such as waveforms indicating the ECG data and/or impedance data, notifications about detected heart rhythms, and the like.

### Example Clauses

1. A dual-mode external defibrillator, including: a detection circuit configured to detect an electrocardiogram (ECG) of a subject; a discharge circuit configured to output an electrical shock to the subject; and a processor configured to: determine that the subject is operating the dual-mode external defibrillator; in response to determining that the subject is operating the dual-mode external defibrillator: determine that the ECG is indicative of ventricular fibrillation (VF); in response to determining that the ECG is indicative of VF, determine that the subject is unconscious; and in response to determining that the subject is unconscious, cause the discharge circuit to output the electrical shock to the subject.

2. The dual-mode external defibrillator of clause 1, further including: an electrical contact configured to be touched by the subject, wherein the detection circuit is further configured to detect an electrical potential between the electrical contact and electrodes affixed to the subject, the ECG being an electrical potential between the electrodes, and wherein the processor is configured to determine that the subject is operating the dual-mode external defibrillator by analyzing the electrical potential between the electrical contact and the electrodes affixed to the subject.

3. The dual-mode external defibrillator of clause 1 or 2, further including: an input device configured to receive an input signal from the subject, wherein the processor is configured to determine that the subject is operating the dual-mode external defibrillator by identifying an artifact in the ECG that corresponds to a time at which the input signal is received from the subject.

4. A medical device, including: a detection circuit configured to detect an electrocardiogram (ECG) of a subject; a therapy component configured to output a therapy to the subject; and a processor configured to: determine that the subject is operating the medical device; in response to determining that the subject is operating the medical device: determine that the subject is incapacitated; and in response to determining that the subject is incapacitated, causing the therapy component to output the therapy to the subject.

5. The medical device of clause 4, wherein the therapy includes defibrillation, pacing, chest compressions, assisted ventilation, or administration of a medication.

6. The medical device of clause 4 or 5, further including: an electrical contact electrically coupled to the detection circuit, the detection circuit being configured to detect an electrical potential between the electrical contact and an electrode affixed to the subject, and wherein the processor is configured to determine that the subject is incapacitated by: analyzing the electrical potential between the electrical contact and the electrode.

7. The medical device of one of clauses 4 to 6, further including: an input device configured to receive an input signal from the subject, wherein the processor is configured to determine that the subject is operating the medical device by: determining a time at which the input signal is received from the subject; and identifying a motion artifact in the ECG corresponding to the time.

8. The medical device of one of clauses 4 to 7, further including: a sensor configured to detect a physiological parameter of the subject, wherein the processor is configured to determine that the subject is incapacitated by analyzing the physiological parameter.

9. The medical device of clause 8, wherein the physiological parameter includes a plethysmograph of the subject, and wherein the processor is configured to determine that the subject is incapacitated by determining that the plethysmograph indicates that the subject lacks pulsatile blood flow.

10. The medical device of clause 8 or 9, wherein the physiological parameter includes an acceleration of a limb of the subject, and wherein the processor is configured to determine that the subject is incapacitated by determining that the acceleration is below a threshold acceleration for greater than a threshold period of time.

11. The medical device of one of clauses 4 to 10, wherein the processor is configured to cause the therapy component to output the therapy to the subject by: determining that the ECG is indicative of ventricular fibrillation, ventricular tachycardia, or bradycardia; and in response to determining that the ECG is indicative of the arrhythmia, causing the therapy component to output an electrical shock or pace pulses to the heart of the subject.

12. The medical device of one of clauses 4 to 11, further including: an input device configured to detect an input signal from the subject, wherein the processor is configured to determine that the subject is incapacitated by: determining that the input signal has ceased.

13. A method, including: detecting an electrocardiogram (ECG) of a subject; determining that the subject is operating a medical device; in response to determining that the subject is operating the medical device: determine that the subject is incapacitated; and in response to determining that the subject is incapacitated, outputting a therapy to the subject.

14. The method of clause 13, wherein the therapy includes defibrillation, pacing, chest compressions, assisted ventilation, or administration of a medication.

15. The method of clause 13 or 14, wherein determining that the subject is operating the medical device includes analyzing the ECG.

16. The method of one of clauses 13 to 15, further including: detecting an electrical potential between an electrical contact and an electrode affixed to the subject, wherein determining that the subject is operating the medical device includes analyzing an electrical potential between the electrical contact and the electrode.

17. The method of one of clauses 13 to 16, further including: receiving an input signal from the subject; determining a time at which the input signal is received from the subject, wherein determining that the subject is operating the medical device includes identifying an artifact in the ECG corresponding to the time.

18. The method of one of clauses 13 to 17, wherein outputting the therapy to the subject is in response to determining that the ECG is indicative of ventricular fibrillation, ventricular tachycardia, or bradycardia.

19. The method of clause 18, wherein the therapy is output at a predetermined time period after the ECG is indicative of the ventricular fibrillation, ventricular tachycardia, or bradycardia.

20. The method of one of clauses 13 to 19, further including: detecting an input signal from the subject, wherein determining that the subject is incapacitated includes determining that the input signal has ceased.

21. A dual-mode external defibrillator, including: a detection circuit configured to detect an electrocardiogram (ECG) of a subject; a discharge circuit configured to output a first electrical shock and a second electrical shock to the subject; an input device configured to receive an input signal from a rescuer; and a processor configured to: activate a subject-operated mode; in response to activating the subject-operated mode: determine that a first segment of the ECG is indicative of ventricular fibrillation (VF); in response to determining that the first segment of the ECG is indicative of VF, determine that the subject is unconscious; in response to determining that the subject is unconscious, cause the discharge circuit to output the first electrical shock to the subject; activate a rescuer-operated mode; in response to activating the rescuer-operated mode: determine that a second segment of the ECG is indicative of VF; in response to determining that the second segment of the ECG is indicative of VF, outputting a recommendation to administer a second electrical shock to the subject; and in response to the input device receiving the input signal, cause the discharge circuit to output the second electrical shock to the subject.

22. The dual-mode external defibrillator of clause 21, the input signal being a first input signal, wherein the input device is configured to receive a second input signal from the rescuer, and wherein the processor is configured to activate the rescuer-operated mode by determining that a motion artifact in the ECG is absent at a time at which the input device receives the second input signal.

23. The dual-mode external defibrillator of clause 21 or 22, wherein the processor is further configured to: in response to activating the subject-operated mode and causing the discharge circuit to output the first electrical shock to the subject: determine that the subject has not regained consciousness within a threshold period of time after the electrical shock is output to the subject by the discharge circuit; and in response to determining that the subject has not regained consciousness within the threshold period of time after the electrical shock is output to the subject by the discharge circuit, cause the discharge circuit to administer pace pulses to the subject.

24. A medical device, including: a detection circuit configured to detect a physiological parameter of a subject; a therapy component configured to output a first therapy to the subject; and a processor configured to: activate a subject-operated mode; in response to activating the subject-operated mode: determine that the physiological parameter is indicative of a condition during a first time period; in response to determining that the physiological parameter is indicative of the condition during the first time period, determine that the subject is unconscious; in response to determining that the subject is unconscious, cause the therapy component to output the first therapy to the subject; activate a rescuer-operated mode; and in response to activating the rescuer-operated mode, output a recommendation to administer a second therapy to the subject.

25. The medical device of clause 24, wherein the physiological parameter includes an electrocardiogram (ECG), a heart rate, a capnograph, a partial pressure of carbon dioxide, a respiratory rate, a blood pressure, a blood oxygenation, a transthoracic impedance, a blood velocity, or a temperature.

26. The medical device of clause 24 or 25, wherein the condition includes ventricular fibrillation, ventricular tachycardia, bradycardia, asystole, hypoxemia, lack of pulsatile flow, seizure, or apnea.

27. The medical device of one of clauses 24 to 26, wherein the first therapy includes a first electrical shock, a first set of pace pulses, or first chest compressions, and wherein the second therapy includes a second electrical shock, a second set of pace pulses, or second chest compressions.

28. The medical device of one of clauses 24 to 27, further including: an input device configured to receive an input signal, wherein the processor is configured to activate the subject-operated mode or the rescuer-operated mode in response to the input device receiving the input signal.

29. The medical device of clause 28, wherein the processor is further configured to: detect a motion artifact in the physiological parameter at a particular time; and determine that the input signal is received by the input device at the particular time, wherein the processor is configured to activate the subject-operated mode in response to detecting the motion artifact at the particular time and determining that the input signal is received by the input device at the particular time.

30. The medical device of clause 28 or 29, wherein the processor is further configured to: detect an absence of a motion artifact in the physiological parameter at a time at which the input signal is received by the input device, wherein the processor is configured to activate the rescuer-operated mode in response to determining that there is the absence of the motion artifact in the physiological parameter at the time at which the input signal is received by the input device.

31. The medical device of one of clauses 24 to 30, further including: an output device configured to output a report indicating the first therapy, wherein the processor is further configured to: determine that the subject has regained consciousness; and in response to determining that the subject has regained consciousness, cause the output device to output the report.

32. The medical device of one of clauses 24 to 31, wherein the processor is further configured to: in response to activating the subject-operated mode and causing the therapy component to output the first therapy to the subject: determine that the subject has not regained consciousness within a threshold period of time after the first therapy is output to the subject by the therapy component; and in response to determining that the subject has not regained consciousness within the threshold period of time after the first therapy is output is output to the subject by the therapy component, cause the therapy component to output a third therapy to the subject.

33. The medical device of one of clauses 24 to 32, wherein the processor is configured to determine that the subject has not regained consciousness by detecting an absence of a motion artifact in the physiological parameter.

34. A method, including: detecting a physiological parameter of a subject; activating a subject-operated mode of a medical device; in response to activating the subject-operated mode: determining that the physiological parameter is indicative of a condition during a first time period; in response to determining that the physiological parameter is indicative of the condition during the first time period, determining that the subject is unconscious; and in response to determining that the subject is unconscious, outputting a first therapy to the subject; activating a rescuer-operated mode; and in response to activating the rescuer-operated mode, outputting a recommendation to administer a second therapy to the subject.

35. The method of clause 34, wherein the physiological parameter includes an electrocardiogram (ECG), a heart rate, a capnograph, a partial pressure of carbon dioxide, a respiratory rate, a blood pressure, a blood oxygenation, a transthoracic impedance, or a temperature.

36. The method of clause 34 or 35, wherein the condition includes ventricular fibrillation, ventricular tachycardia, bradycardia, asystole, hypoxemia, lack of pulsatile flow, seizure, or apnea.

37. The method of one of clauses 34 to 36, wherein the first therapy includes a first electrical shock, first paces, or first chest compressions, and wherein the second therapy includes a second electrical shock, second paces, or second chest compressions.

38. The method of one of clauses 34 to 37, further including: receiving an input signal at a particular time; and detecting a motion artifact in the physiological parameter at the particular time, wherein activating the subject-operated mode is in response to receiving the input signal at the particular time and detecting the motion artifact at the particular time.

39. The method of one of clauses 34 to 38, further including: receiving an input signal at a particular time; and detecting an absence of a motion artifact in the physiological parameter at the particular time, wherein activating the rescuer-operated mode is in response to receiving the input signal at the particular time and detecting the absence of the motion artifact in the physiological parameter at the particular time.

40. The method of one of clauses 34 to 39, further including: in response to activating the subject-operated mode and outputting the first therapy to the subject: determining that the subject has not regained consciousness within a threshold period of time after the first therapy is output to the subject; and in response to determining that the subject has not regained consciousness within the threshold period of time after the first therapy is output is output to the subject, outputting a third therapy to the subject.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; 18% of the stated value; 17% of the stated value; 16% of the stated value; 15% of the stated value; 14% of the stated value; ±13% of the stated value; 12% of the stated value; 11% of the stated value; 10% of the stated value; 9% of the stated value; 8% of the stated value; 7% of the stated value; 6% of the stated value; 5% of the stated value; 4% of the stated value; 3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A dual-mode external defibrillator, comprising:
a detection circuit configured to detect an electrocardiogram (ECG) of a subject;
a discharge circuit configured to output an electrical shock to the subject; and
a processor configured to:
determine that the subject is operating the dual-mode external defibrillator;
in response to determining that the subject is operating the dual-mode external defibrillator:
determine that the ECG is indicative of ventricular fibrillation (VF);
in response to determining that the ECG is indicative of VF, determine that the subject is unconscious; and
in response to determining that the subject is unconscious, cause the discharge circuit to output the electrical shock to the subject.

2. The dual-mode external defibrillator of claim 1, further comprising:
an electrical contact configured to be touched by the subject,
wherein the detection circuit is further configured to detect an electrical potential between the electrical contact and electrodes affixed to the subject, the ECG being an electrical potential between the electrodes, and
wherein the processor is configured to determine that the subject is operating the dual-mode external defibrillator by analyzing the electrical potential between the electrical contact and the electrodes affixed to the subject.

3. The dual-mode external defibrillator of claim 1, further comprising:
an input device configured to receive an input signal from the subject,
wherein the processor is configured to determine that the subject is operating the dual-mode external defibrillator by identifying an artifact in the ECG that corresponds to a time at which the input signal is received from the subject.

4. A medical device, comprising:
a detection circuit configured to detect an electrocardiogram (ECG) of a subject;
a therapy component configured to output a therapy to the subject; and
a processor configured to:
determine that the subject is operating the medical device;
in response to determining that the subject is operating the medical device:
determine that the subject is incapacitated; and
in response to determining that the subject is incapacitated, causing the therapy component to output the therapy to the subject.

5. The medical device of claim 4, wherein the therapy comprises defibrillation, pacing, chest compressions, assisted ventilation, or administration of a medication.

6. The medical device of claim 4, further comprising:
an electrical contact electrically coupled to the detection circuit, the detection circuit being configured to detect an electrical potential between the electrical contact and an electrode affixed to the subject, and
wherein the processor is configured to determine that the subject is incapacitated by:
analyzing the electrical potential between the electrical contact and the electrode.

7. The medical device of claim 4, further comprising:
an input device configured to receive an input signal from the subject,
wherein the processor is configured to determine that the subject is operating the medical device by:
determining a time at which the input signal is received from the subject; and
identifying a motion artifact in the ECG corresponding to the time.

8. The medical device of claim 4, further comprising:
a sensor configured to detect a physiological parameter of the subject,
wherein the processor is configured to determine that the subject is incapacitated by analyzing the physiological parameter.

9. The medical device of claim 8, wherein the physiological parameter comprises a plethysmograph of the subject, and
wherein the processor is configured to determine that the subject is incapacitated by determining that the plethysmograph indicates that the subject lacks pulsatile blood flow.

10. The medical device of claim 8, wherein the physiological parameter comprises an acceleration of a limb of the subject, and
wherein the processor is configured to determine that the subject is incapacitated by determining that the acceleration is below a threshold acceleration for greater than a threshold period of time.

11. The medical device of claim 4, wherein the processor is configured to cause the therapy component to output the therapy to the subject by:
determining that the ECG is indicative of an arrhythmia comprising ventricular fibrillation, ventricular tachycardia, or bradycardia; and
in response to determining that the ECG is indicative of the arrhythmia, causing the therapy component to output an electrical shock or pace pulses to a heart of the subject.

12. The medical device of claim 4, further comprising:
an input device configured to detect an input signal from the subject,
wherein the processor is configured to determine that the subject is incapacitated by:
determining that the input signal has ceased.

13. A method, comprising:
detecting an electrocardiogram (ECG) of a subject;
determining that the subject is operating a medical device;
in response to determining that the subject is operating the medical device:
determine that the subject is incapacitated; and
in response to determining that the subject is incapacitated, outputting a therapy to the subject.

14. The method of claim 13, wherein the therapy comprises defibrillation, pacing, chest compressions, assisted ventilation, or administration of a medication.

15. The method of claim 13, wherein determining that the subject is operating the medical device comprises analyzing the ECG.

16. The method of claim 13, further comprising:
detecting an electrical potential between an electrical contact and an electrode affixed to the subject,
wherein determining that the subject is operating the medical device comprises analyzing an electrical potential between the electrical contact and the electrode.

17. The method of claim 13, further comprising:
receiving an input signal from the subject;
determining a time at which the input signal is received from the subject,
wherein determining that the subject is operating the medical device comprises identifying an artifact in the ECG corresponding to the time.

18. The method of claim 13, wherein outputting the therapy to the subject is in response to determining that the ECG is indicative of an arrhythmia comprising ventricular fibrillation, ventricular tachycardia, or bradycardia.

19. The method of claim 18, wherein the therapy is output at a predetermined time period after the ECG is indicative of the arrhythmia.

20. The method of claim 13, further comprising:
detecting an input signal from the subject,
wherein determining that the subject is incapacitated comprises determining that the input signal has ceased.
